SEQUENCE LISTING (4 sequences)

SEQ ID NO: 1 - DNA, 24 bp, artificial sequence
  primer_binding: ALOX5 Forward primer
agccaacaagattgttcccatcgc

SEQ ID NO: 2 - DNA, 24 bp, artificial sequence
  primer_binding: ALOX5 Reverse primer
tggcaataccgaacacctcagaca

SEQ ID NO: 3 - DNA, 20 bp, artificial sequence
  primer_binding: GADPH sense primer
acccccaatgtatccgttgt

SEQ ID NO: 4 - DNA, 20 bp, artificial sequence
  primer_binding: GADPH antisense primer
tactccttggaggccatgta

(19) United States
(12) Patent Application Publication
(10) Pub. No.: US 2009/0042849 A1
(43) Pub. Date: Feb. 12, 2009

(54) Phosphorylation of 5-lipoxygenase at ser523 and uses thereof

(76) Inventor: Yochai Birnbaum, Houston, TX (US)

Correspondence Address: Benjamin Aaron Adler;ADLER & ASSOCIATES, 8011 Candle Lane, Houston, TX, 77071, US

(21) Appl. No.: 11/999,534

(22) Filed: Dec. 6, 2007

Related U.S. Application Data
(60) Provisional application No. 60/873,100, filed on Dec. 6, 2006.

(51) Int. Cl. A61K 31/555; A61K 31/495; C12Q 1/26; A61P 9/00; A61K 31/425; C12Q 1/48

(52) U.S. Cl. 514/184; 514/249; 435/25; 435/15; 514/369

(57) ABSTRACT

The present invention provides novel mechanisms that regulate the production of anti-inflammatory and pro-inflammatory mediators generated by 5-lipoxygenase. In this regard, the present invention establishes that phosphorylation of 5-Lipoxygenase by protein kinase A, has a crucial role in determining the end products of 5-Lipoxygenase. With translocation to the nucleus, potent proinflammatory leukotrienes are produced, whereas following phosphorylation by protein kinase A, anti-inflammatory mediators are produced. The present invention also discloses compounds that regulate these pro- and anti-inflammatory mediators.

### CROSS REFERENCE TO RELATED APPLICATION

This non-provisional application claims benefit of provisional application U.S. Ser. No. 60/873,100 filed on Dec. 6, 2006, now abandoned.

### BACKGROUND OF THE INVENTION

1. Field of the Invention

The present invention relates generally to the field of cardiology. More specifically the present invention relates to the regulation of 5-Lipoxygenase by phosphorylation via protein kinase A activation for the production of anti-inflammatory mediators to augment the anti-inflammatory effects and reduce the side-effects of HMGCoA Reductase Inhibitors (statins) and/or thiazolidines.

2. Description of the Related Art

Both the perioxisome proliferator-activated receptors (PPAR-γ) agonist pioglitazone [5-[[4-[2-(5-ethylpyridin-2 yl)ethoxy]phenyl]methyl]thiazolidine-2,4-dione] (PIO) [1,2] and the 3-hydroxy-3-methylglutaryl coenzyme A (HMGCoA) reductase inhibitor atorvastatin [7-[2-(4-fluorophenyl)-5-(1-methylethyl)-3-phenyl-4-(phenylcarbamoyl)-1H-pyrrol-1-yl]-3,5-dihydroxy-heptanoic acid] (ATV) [3,4] have anti-inflammatory properties, reducing serum markers of inflammation including C-reactive protein, IL-6 and TNF-a. However, the underlying mechanisms of their anti-inflammatory properties are unknown.

Recent studies have demonstrated that both PIO and ATV increase the production of 15-epi-lipoxin A₄ [(5S,6R,15R)-5,6,15-trihydroxy-7,9,13-trans-1 1-ciseicosatetraenoic Acid] (15ELXA), a lipid mediator with strong anti-inflammatory properties [5]. 15ELXA has been shown to inhibit the production of IL-6 and TNF-a [6,9]. 15ELXA is a product of cycloxygenase-2 (COX2) and 5-lipoxygenase (5LO) [5]. However, 5-lipoxygenase also catalyzes the oxygenation of arachidonic acid (AA) to 5-HPETE [5(S)-hydroperoxy-6-trans-8,11,14-9-eicosatetraenoic acid], and the further dehydration to the allylic epoxide leukotriene A₄, the initial reactions in leukotriene (LT) biosynthesis. Leukotrienes are lipid mediators with strong pro-inflammatory properties [10]. Until recently, it was thought that 5-lipoxygenase is expressed mainly in inflammatory cells [polymorphonuclear leukocytes, monocytes/macrophages, mast cells, B-lymphocytes, dendritic cells, and foam cells in human atherosclerotic tissue] [10]. It has been previously shown that 5-lipoxygenase is expressed in rat cardiomyocytes [5]. Upon cell activation leading to leukotriene biosynthesis, 5-lipoxygenase and cytosolic phospholipase A₂ (cPLA₂) a generator of AA from the membrane phospholipids migrate to the perinuclear membrane [10-12]. This shift involves Ca²⁺-induced binding of the C2-like domain to phospholipids [10]. Luo et al reported that phosphorylation of 5-lipoxygenase at Ser⁵²³ by protein kinase A (PKA) prevents the migration of 5-lipoxygenase to the perinuclear membrane, resulting in decreased synthesis of leukotriene [13]. Statins have been shown to activate protein kinase A [14.15].

5-lipoxygenase produces both 15-epilipoxin-A₄, an anti-inflammatory mediator and leukotriene B₄, a pro-inflammatory mediator. The prior art is deficient in the knowledge of mechanisms that regulate the production of one or the other mediator. Specifically, the prior art is lacking in the knowledge of the role of protein kinase A mediated phosphorylaion at Ser⁵²³ of 5-lipoxygenase and its involvement in production of anti-inflammatory (15-epiloxin-A₄) or pro-inflammatory (leukotriene-B4) mediators. Further, the prior art is deficient in the knowledge of the mechanism of anti-inflammatory properties of ATV and PIO and whether 5-lipoxygenase phosphorylation is crucial for the anti-inflammatory action of these drugs. The instant invention fulfills this long-standing need and desire in the art.

### SUMMARY OF THE INVENTION

The present invention is directed to a method of attenuating a pro-inflammatory state specific for a disease in an individual. Such a method comprises administering a pharmacologically effective dose of a compound(s) that either phosphorylates 5-lipoxygenase or augments the phosphorylation of 5LO by protein kinase A, prevents translocation of the 5-lipoxygenase from cytosol to the peri-nuclear membrane or both. Administration of such compound(s) attenuates the pro-inflammatory state specific for the disease in the individual.

The present invention is also directed to a method of decreasing the risk or progression of a disease in an individual. Such a method comprises administering a pharmacologically effective dose of a compound that inhibits the production of Leukotriene-B₄ to the individual. Administration of such compound(s) decreases the risk or progression of a disease in the individual.

The present invention is further directed to a method of augmenting anti-inflammatory effects in an individual in need of such augmentation. Such a method comprises administering a pharmacologically effective dose of a compound that phosphorylates or augments phosphorylation of serine-523 residue of 5-Lipoxygenase such the phosphorylation of 5-Lipoxygenase regulates the production of anti-inflammatory and pro-inflammatory metabolites of arachidonic acid thereby augmenting the anti-inflammatory effects in said individual.

The present invention is also directed to a method for screening for a drug useful for augmenting anti-inflammatory effects in a disease state. Such a method comprises contacting a sample peptide comprising of the serine-523 residue of 5-Lipoxygenase with a test compound and providing the necessary enzymes and ATP. The effect of the compound on the phosphorylation of the serine-523 residue is then determined, where phosphorylation of the serine-523 residue of the peptide in the presence of the test compound indicates that the test compound is the drug useful for augmenting anti-inflammatory effects in said disease state.

The present invention is also directed to a method of ameliorating the side effects of statin therapy in an individual. Such a method comprises administering pharmacologically effective amounts of of a protein kinase A activator or a compound that directly phosphorylates 5LO in combination with statins and/or thiazolidinediones. The administration of these compounds ameliorates the side-effects of statin therapy in the individual.

The present invention is further directed to a method of inducing myocardial protection in an individual. Such a method comprises administrating pharmacologically effective amounts of a protein kinase A activator or a compound that directly phosphorylates 5LO in combination with statins and/or thiazolidinediones. The administration of these compounds synergistically reduces the infarct size, thereby inducing myocardial protection in the individual.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention as well as others which will become clear are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIGS. 1A-1D show the effect of PIO and ATV alone or with H-89, PKA inhibitor, on myocardial levels of total 5LO and P-5LO. FIGS. 1A-1B show representative immunoblots of 5-lipoxygenase and the corresponding b-actin in the rat heart cytosolic fraction (FIG. 1A) and nuclear fraction (FIG. 1B). FIGS. 1C-1D show densitometric analyses of 5-lipoxygenase expression (as a percent of the sham 5LO/b-actin ratio) in the cytosolic (FIG. 1C) and nuclear fraction (FIG. 1D). P<0.001 for the differences among groups in both the cytosolic and nuclear fraction. *—p<0.005 versus sham; †—p<0.001 versus PIO+ATV; #—p<0.001 versus LPS.

FIGS. 2A-2B compares the effect of PIO and ATV to that of LPS on 5LO phosphroylation. FIG. 2A shows representative immunoblots of P-5LO and the corresponding b-actin in the rat hearts. FIG. 2B shows densitometric analyses of P-LO expression (as a percent of the sham 5LO/b-actin ratio). P<0.001 for the differences among groups. *—p<0.001 versus sham; #—p<0.001 versus PIO+ATV.

FIG. 3 demonstrates immunofluorescence of 5-lipoxygenase (red), myosin (green) and DAPI (blue) of myocardium of rat treated with PIO+ATV, LPS or sham. In the PIO+ATV treated rat, there is increased expression of 5-lipoxygenase in the cytosol of cells stained positive for myosin (cardiomyocytes). In the LPS treated rat, the 5-lipoxygenase is expressed around the nuclei of the cardiomyocytes (magnification ×120).

FIGS. 4A-4D show immunofluorescence of 5-lipoxygenase (red), myosin (green) and DAPI (blue) of adult rat cardiomyocytes treated with vehicle (FIG. 4A); PIO+ATV (FIG. 4B), PIO+ATV+H-89 (FIG. 4C), and H-89 (FIG. 4D). PIO+ATV increased the expression of 5-lipoxygenase in the cytosole. H-89 alone had no effect; however, when combined with PIO+ATV it was associated with translocation of 5-lipoxygenase towards the perinuclear membrane (magnification ×40).

FIG. 5 shows confocal microscopy (magnification ×120) of a cardiac myocyte treated with PIO+ATV+H-89. There is localization of the 5-lipoxygenase (red) around the nuclear membrane, but not inside the nucleus (blue).

FIGS. 6A-6D shows immunofluorescence of 5-lipoxygenase (red), cPLA₂ (green) and DAPI (blue) of adult rat cardiac myocytes treated with vehicle (FIG. 6A); PIO+ATV (FIG. 6B), PIO+ATV+H-89 (FIG. 6C), and H-89 (FIG. 6D). PIO+ATV increased the expression of 5-lipoxygenase in the cytosole. PIO+ATV also increased the expression of cPLA₂ with some predilection towards the perinuclear zone. H-89 alone had no effect on 5-lipoxygenase and cPLA₂ expression; however, when combined with PIO+ATV it resulted in translocation of 5LO towards the perinuclear membrane (magnification ×40).

FIGS. 7A-7D demonstrate immunofluorescence of 5LO (red), COX2 (green) and DAPI (blue) of adult rat cardiac myocytes treated with vehicle (FIG. 7A); PIO+ATV (FIG. 7B), PIO+ATV+H-89 (FIG. 7C), and H-89 (FIG. 7D). PIO+ATV increased the expression of 5-lipoxygenase and COX2 in the cytoplasm. H-89 alone had no effect on 5-lipoxygenase and COX2 expression. In the PIO+ATV+H-89 group 5-lipoxygenase staining is enhanced around the nucleus. In contrast, H-89 did not affect the increased expression of COX2 by PIO+ATV (magnification ×40).

FIGS. 8A-8B shows effect of PIO+ATV on 5LO and p-5LO levels. FIG. 8A shows representative immunoblots of P-5-lipoxygenase and the corresponding b-actin in rat adult cardiac myocytes. FIG. 8B shows densitometric analyses of P-5-lipoxygenase expression (as a percent of the sham P-5LO/b-actin ratio). P<0.001 for the difference among groups. *—p<0.001 versus PIO+ATV; p<0.005 versus sham; †—p=0.009 versus PIO+ATV+H-89.

FIGS. 9A-9B show levels of 15ELXA and LTB4 levels in adult rat cardiac myocytes treated with PIO+ATV, H-89 and their combination. FIG. 9A shows levels of 15ELXA and FIG. 9B shows levels of LTB4 in adult rat cardiac myocytes treated with PIO+ATV, H-89, and their combination. P<0.001 for the overall difference among groups. *—p<0.001 versus sham; #—p<0.001 versus PIO+ATV; †—p<0.001 versus PIO+ATV+H-89.

FIG. 10 shows co-immunoprecipitation of the whole cell lysate. 5LO precipitated with COX2 in the ATV and PIO group; whereas 5LO precipitated with cPLA₂ in the ATV+H-89 and PIO+H-89 groups.

FIG. 11 shows co-immunoprecipation of 5LO with COX2 and cPLA2 in the cytosolic and membranous fractions. In both the ATV and PIO group 5LO precipitated with COX2 in the cytosolic, but not the membranous fraction.

FIG. 12 shows rtPCR of 5LO mRNA. There is expression of 5LO in both adult rat cardiomyocytes and White Blood Cells. PIO, ATV and H-89 alone or in combination did not affect 5LO expression. In contrast, LPS significantly increased 5LO expression in the White Blood Cells. *—p<0.001 versus control.

FIGS. 13A-13B show schematic presentation of the interaction between P-5LO and COX2 in the cytosole, resulting in the production of 15ELXA, with PIO and ATV treatment. However, when PKA is blocked by H-89, 5LO is interacting with cPLA2 on the membranes, leading to formation of LT from AA.

FIG. 14 shows synergistic effect of cilostazol (Pletal) and atorvastatin on myocardial protection. ATV at 2 mg/kg/d had no effect. Cilostazol 20 mg/kg/d reduced infarct size. However, when combined with ATV 2 mg/kg/d the effect was much greater. Infarct size in the ATV 2 mg/kg/d (30.48±1.46%) is similar to the controls (33.97±2.76%). The cilostazol group(15.47±1.61%) is significantly smaller than the controls (p<0.001). The combination (4.31±0.48%) is significantly different (<0.001) from the controls and ATV 2 mg/kg and (=0.006) versus the cilostazol alone group.

### DETAILED DESCRIPTION OF THE INVENTION

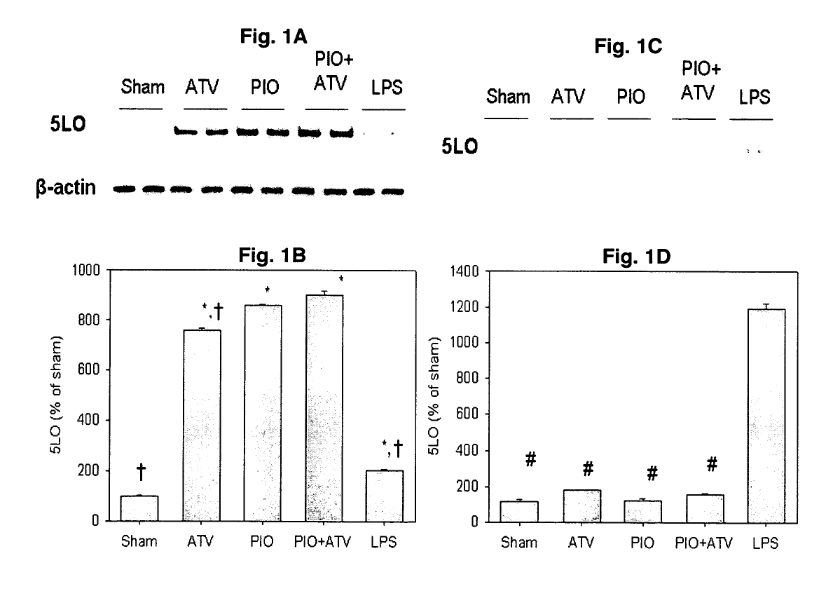

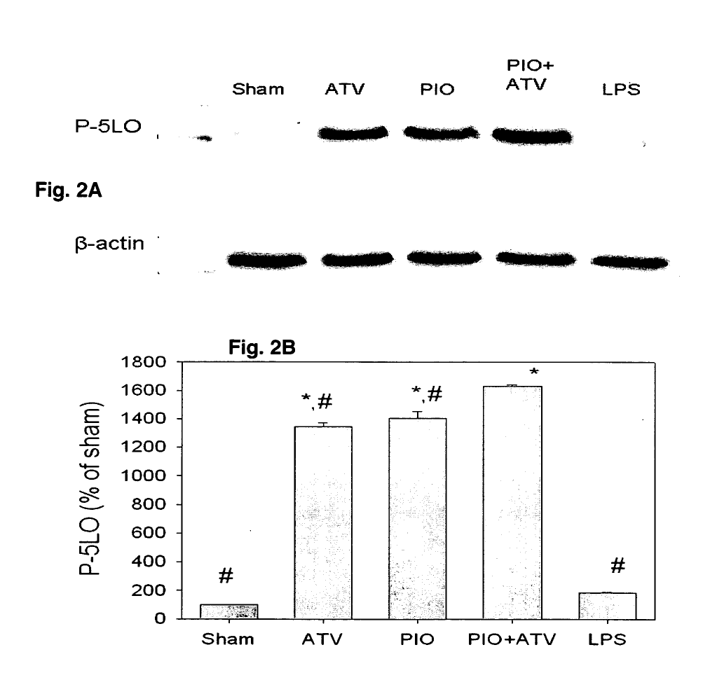

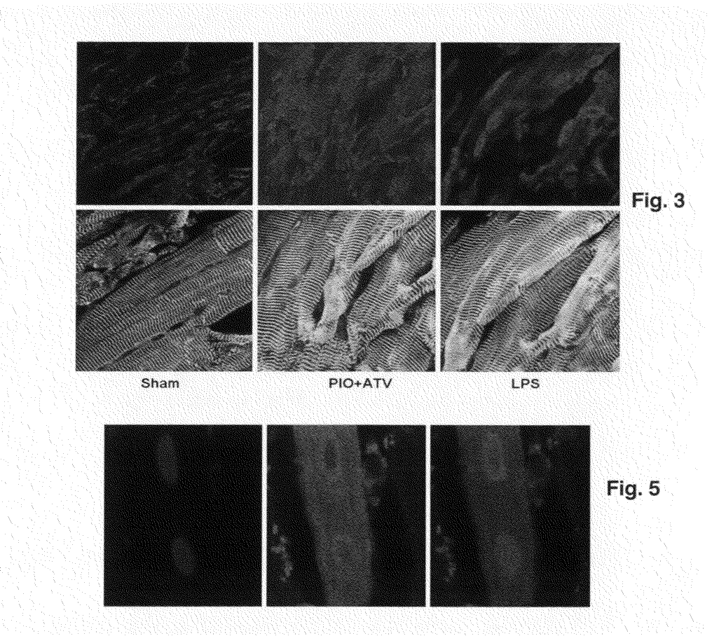

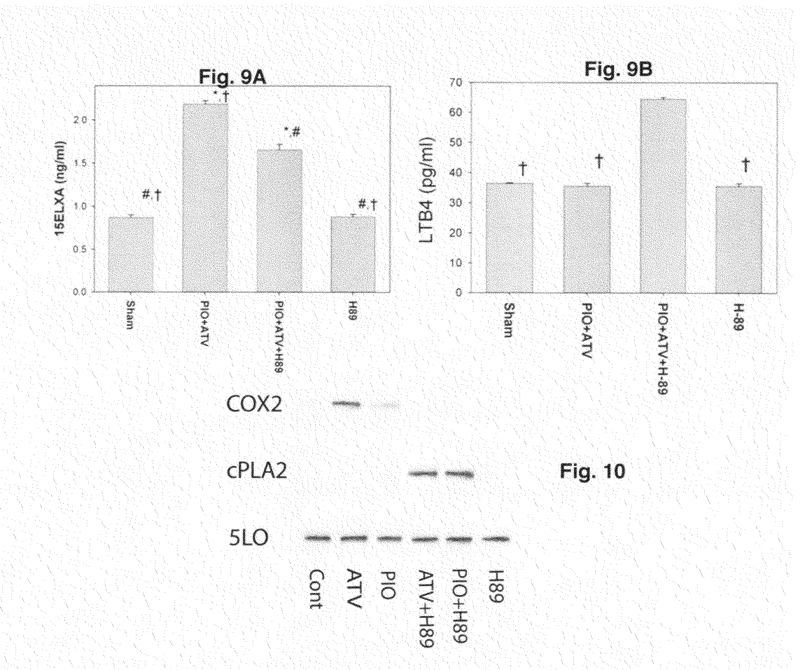

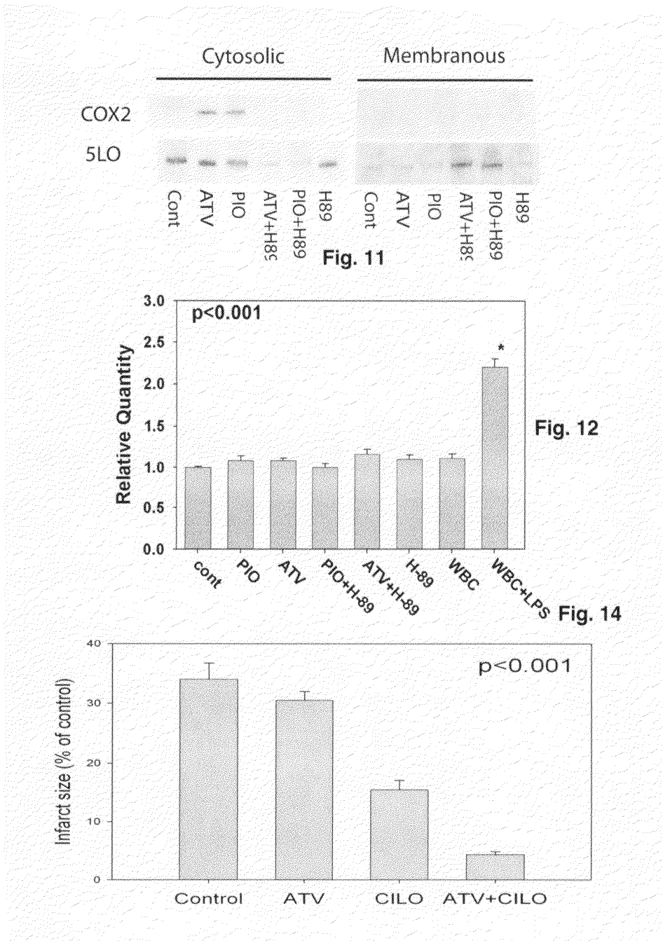

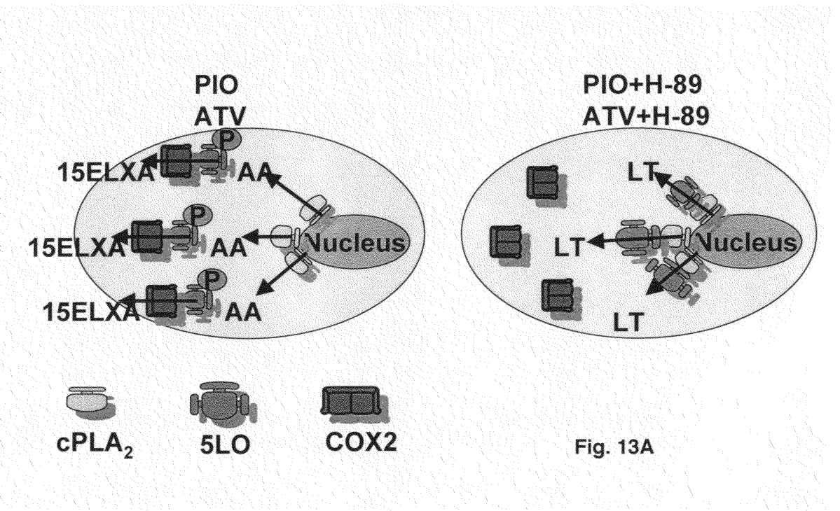

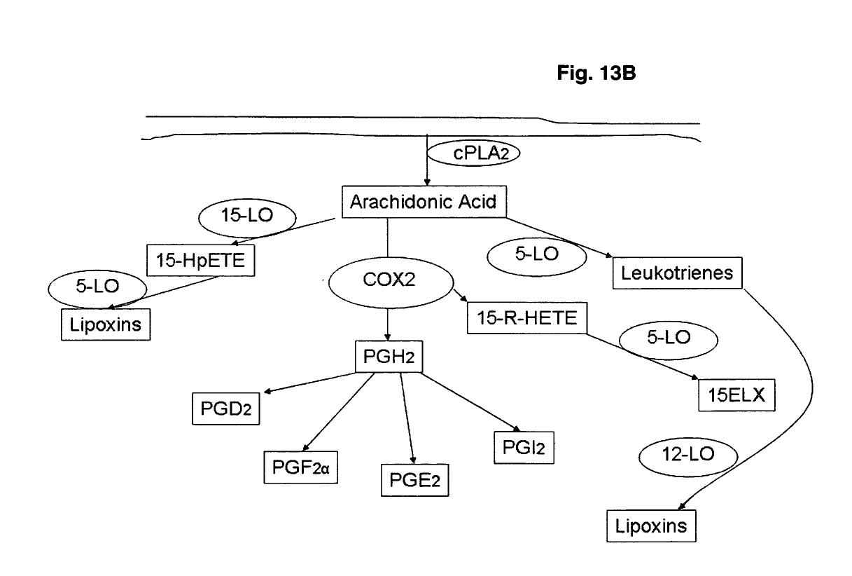

The instant invention demonstrates that both PIO and ATV augment 5-lipoxygenase phosphorylation at Ser⁵²³ in the rat myocardium and that this effect was blocked by H-89, a PKA inhibitor (FIG. 1). In contrast, the pro-inflammatory stimulatio with LPS caused less Ser⁵²³ phosphorylation of 5LO (FIG. 2). Both PIO and ATV caused a small increase in 5LO levels in the cytosolic fraction (FIG. 3) without detectable change in total 5LO levels (FIG. 1), suggesting translocation of 5LO into the cytosolic fraction. In contrast, -inhibiting protein kinase A with H-89 prevented the Ser⁵²³ phosphorylation of 5-lipoxygenase by PIO and ATV (FIG. 1) and caused a shift of 5-lipoxygenase to the membranous fraction. Under these conditions 5-lipoxygenase co-immunoprecipitated with cPLA2 (FIG. 10) and metabolized arachidonic acid, generated by cPLA₂, into LTB4, a strong inflammatory mediator. In contrast, when 5-lipoxygenase was prevented from shifting by Ser⁵²³ phosphorylation, it interacted with COX2 in the cytosolic fraction (FIG. 11) to generate 15ELXA (FIG. 9A), a potent anti-inflammatory mediator. Thus, it seems that Ser⁵²³ phosphorylation of 5-lipoxygenase by protein kinase A not only prevents leukotriene (LT) production but also facilitates 15ELXA production and therefore, is a key factor in determining whether the end-products will be pro- or anti-inflammatory mediators. Based on the data presented herein, the present invention provides schematic representation of the interaction between P-5LO and COX2 in the cytosol that results either in the production of pro- or anti-inflammatory mediators (FIGS. 13A-13B).

Until recently, it was thought that 5LO is expressed mainly in inflammatory cells (polymorphonuclear leukocytes, monocytes/macrophages, mast cells, B-lymphocytes, dendritic cells, and foam cells in human atherosclerotic tissue) [10, 25]. However, there is growing evidence that cardiomyocytes participate in innate immunity [26, 27]. It has been shown that cardiomyocytes respond to various injuries by producing some of the mediators that are classically associated with cells of the innate immune system [28]. Massey et al showed that U-70344A, a 5LO inhibitor, prevented the uncoupling of neonatal rat myocardial cells in cultures when exposed to arachidonic acid [29]. Przygodzki et al also showed that MK886, a 5LO inhibitor, prevented calcium ionophore (A23187) induction of reactive oxygen species by neonatal rat cardiomyocytes cultures [30]. Liu et al reported that 5LO and leukotrienes are essential in mediating angiotensin II evoked increases in cytosolic free calcium in neonatal rat cardiomyocytes [31]. Kuzuya et al reported that 5-HPETE production by adult canine cardiac myocytes increases after 45 minutes of ischemia. AA-861, a 5LO inhibitor, attenuated 5-HPETE production and hypoxia-reoxygenation cell injury [32]. These data suggest that cardiomyocytes express active 5LO. It was previously shown that 5LO was expressed in rat cardiomyocytes (5, 33). The present invention demonstrates that rat cardiomyocytes express 5LO mRNA (FIG. 12) and protein (FIG. 1-7).

cPLA₂ is a membrane bound enzyme, generating arachidonic acid from the membrane phospholipids [10-12]. In quiescence cells COX2 is preferentially bound to the nuclear envelope; however, in some cells COX2 can be found inside the nucleus and/or in the endoplasmic reticulum [34]. However, upon stimulation, cytoplasmic accumulation of COX2 has been described in endothelial cells [34, 35]. Interestingly, the present invention demonstrates that the interaction between COX2 and 5LO occurred only in the cytosolic fraction and not in the membranous fraction. In contrast, the interaction between cPLA₂ and 5LO occurred as expected in the membranous fraction. It could be that defects in 5LO phosphorylation could explain the muscle symptoms and/or elevation of muscle and liver enzymes associated with statin therapy. Moreover, it is plausible that in patients with 5LO phosphorylation deficits, statins may not decrease inflammation and thus, may have fewer effects on atherosclerosis.

5LO activating protein (FLAP, also known as ALOX5AP) activates 5LO and facilitate the shift of 5LO to the perinuclear membrane [36. 37]. Increased production of leukotrienes due to gene mutations in 5LO [38], FLAP in Caucasians [39-43] and Japanese [44], and in leukotriene A4 hydroxylase in African American population [45] has been associated with an increased risk of stroke and/or myocardial infarction. However, it is unclear how statins and/or PIO affect arachidonic acid metabolism in patients with these mutations. It might be possible that by Ser⁵²³ phosphorylation of 5LO, statins and PIO prevent 5LO translocation and therefore, attenuate the pro-inflammatory state. On the other hand, it might be possible that these agents cannot prevent this intracellular shift and by upregulating cPLA₂ [17-19] and activating 5LO, augment the production of leukotrienes in patients with such mutations. cPLA₂ generates arachidonic acid, which has been shown to promote activation and translocation of 5LO to the perinuclear membrane [46].

The present invention contemplates exploring this issue in several experimental models. There are also implications in medical fields other than atherosclerosis, as the increased production of leukotrienes by COX2 and 5LO has been implicated with increased risks for colon cancer [47], Alzheimer's disease [48-50] and asthma [51]. As statins may reduce the risk of colon cancer [52] and the progression of Alzheimer's disease [53]; it is plausible that statins (and PIO) may have a role in preventing the membranous shift of 5LO and hence, the production of leukotrienes in various disease states. Phosphorylation as a mechanism responsible for the translocation of apoptotic mediators to the peri-nuclear membrane in response to oxidative stress is not restricted to 5LO; Bcl-2 and Bcl-xL are also phosphorylated and inactivated as anti-apoptotic proteins in response to trauma [54, 55].

Not only inflammatory cells, but also myocardial and endothelial cells can produce arachidonic acid metabolites such as LTB4 and 15ELXA in response to various stimuli. 5LO phosphorylation at Ser⁵²³ by PKA prevents the membranous shift of 5LO and thus, the production of LTB4. Instead, the cytosolic-bound 5LO processes 15-R-HETE, produced by cytosolic COX2, resulting in the production of 15ELXA, a potent anti-inflammatory mediator. Prevention of 5LO translocation towards the perinuclear membrane by protein kinase A mediated phosphorylation at Ser⁵²³ may explain in part the anti-inflammatory and anti-atherosclerosis effects of statins and PIO.

In one embodiment of the present invention, there is provided a method of attenuating a pro-inflammatory state specific for a disease in an individual comprising: administering a pharmacologically effective dose of a compound(s) that phosphorylates 5-lipoxygenase, prevents translocation of the 5-lipoxygenase from cytosol to the peri-nuclear membrane or both, thereby attenuating the pro-inflammatory state specific for the disease in the individual. In such a method, the 5-lipoxygenase may be phosphorylated at serine-523 residue of 5-lipoxygenase. The cytosolic phosphorylated 5-lipoxygenase may interact with Cox-2 to produce 15-epilipoxin-A4. Additionally, compound may directly or indirectly activate protein kinase A such that the activated protein kinase A phosphorylates 5-lipoxygenase. Examples of such compounds may include but are not limited to HMGCoA Reductase inhibitor, Atorvastatin or PPAR-g agonist, pioglitazone, sitagliptin or a combination of thereof. Further, the pro-inflammatory effect may be due to absence of phosphorylation on serine-523, translocation of 5-lipoxygenase to the nuclear membrane, metabolism of arachidonic acid into leukotriene B4 or a combination thereof. Examples of the disease state may include but is not limited to artherosclerosis, arthiritis, asthma, cancer, stroke, myocardial infarction or Alzheimers.

In another embodiment, there is provided a method of decreasing the risk or progression of a disease in an individual comprising: administering a pharmacologically effective dose of a compound that inhibits the production of Leukotriene-B₄ to said individual, thereby decreasing the risk or progression of a disease in the individual. The administration of the compound may result in direct or indirect activation of protein kinase A. Additionally, the activation of protein kinase A might result in phosphorylation of serine-523 residue of 5-Lipoxygenase. Further, this phosphorylation may prevent the localization of 5-Lipoxygenase from cytosol to the perinuclear membrane. Furthermore, the prevention of perinuclear localization might result in decreased leukotriene-B₄ production and an increased 15-epilipoxin-A4 production. Examples of the compound administered herein may include but is not limited to a HMGCoA Reductase inhibitor, Atorvastatin or the PPAR-gagonist, Pioglitazone, sitagliptin, or a combination thereof. Furthermore, examples of the disease state may include but is not limited to artherosclerosis, arthritis, asthma, cancer, stroke, myocardial infarction or Alzheimers.

In yet another embodiment, there is provided a method of augmenting anti-inflammatory effects in an individual in need of such augmentation, comprising: administering a pharmacologically effective dose of a compound that phosphorylates serine-523 residue of 5-Lipoxygenase such said phosphorylation of 5-Lipoxygenase regulates the production of anti-inflammatory and pro-infammatory metabolites of arachidonic acid thereby augmenting the anti-inflammatory effects in said individual. The compound may phosphorylate serine-523 residue of 5-Lipoxygenase by directly or indirectly activating protein kinase A. Further, the phosphorylation of 5-Lipoxygenase may prevent the localization of cytosolic 5-Lipoxygenase to the perinuclear membrane resulting in interaction of phosphorylated 5-lipoxygenase with COX2 and production of anti-inflammatory metabolite of arachidonic acid and inhibition of pro-inflammatory metabolite of arachidonic acid. Examples of the pro-inflammatory metabolite of arachidonic acid may include but is not limited to Leukotriene B₄ and the examples of the anti-inflammatory metabolite of arachidonic acid may include but is not limited to 15-epilipoxin-A₄. Moreover, the 15-epilipoxin-A₄ produced may mediate the anti-inflammatory effects by inhibiting the production of IL-6 and TNF-a. Examples of the compound being administered may include but is not limited to a HMGCoA Reductase inhibitor, Atorvastatin or PPAR-g agonist, Pioglitazone, sitagliptin, or a combination thereof. Further, examples of individual in need of augmentation of anti-inflammatory effect may include but is not limited to those suffering from artherosclerosis, arthritis, asthma, cancer, stroke, myocardial infarction or Alzheimer's.

In yet another embodiment, there is provided a method for screening for a drug useful for augmenting anti-inflammatory effects in a disease state comprising: contacting a sample peptide comprising the serine-523 residue of 5-Lipoxygenase with a test compound; providing the necessary enzymes and ATP, and determining the effect of the compound on the phosphorylation of the serine-523 residue, wherein phosphorylation of the serine-523 residue of the peptide in the presence of the test compound indicates that the test compound is the drug useful for augmenting anti-inflammatory effects in said disease state. Examples of the drug may include but is not limited to a drug that is an activator of protein kinase A, that prevents localization of 5-lipoxygenase to the peri-nuclear membrane or both. Examples of the disease state may include but is not limited to artherosclerosis, arthiritis, asthma, cancer, stroke, myocardial infarction or Alzheimers.

In yet another embodiment, there is provided a method of ameliorating the side effects of statin therapy in an individual comprising: administering pharmacologically effective amounts of a protein kinase A activator in combination with statins and/or thiazolidinediones, where the administration ameliorates the side-effects of statin therapy in the individual. The side effects may comprise muscle aches and/or elevation of muscle and/or liver enzymes. The administration of protein kinase A activator may lead to phosphorylation of serine-523 residue on 5-lipoxygenase and may prevent translocation of said phosphorylated 5-Lipoxygenase from the cytosol to perinuclear membrane, such that the phosphorylated cytosolic 5-Lipoxygenase may interact with COX-2, producing 1 5-epilipoxin A4 and inhibiting the production of Leukotriene-B4. Additionally, the production of the 15-epilipoxin-A₄ may inhibit the production of IL-6 and TNF-a. Examples of the individual on statin therapy may include but is not limited to those suffering from artherosclerosis, arthritis, asthma, cancer, stroke, myocardial infarction or Alzheimer's.

In still yet another embodiment of the present invention there is provided a method of inducing myocardial protection in an individual comprising: administrating pharmacologically effective amounts of a protein kinase A activator in combination with statins and/or thiazolidinediones, where the administration synergistically reduces the infarct size, thereby inducing myocardial protection in the individual. The protein kinase A activator may increase the intracellular levels of cyclic adenosine monophosphate (cAMP) such that said increased cAMP levels activates protein kinase A. Examples of such protein kinase A activators may include but is not limited to cilostazol or sitagliptin. Additionally, examples of statin may include but is not limited to—Atrovastatin, and the examples of thiazolidinedione may include but is not limited to pioglitazone. Further, the individual may be suffering from a myocardial infarction.

As used herein, the term, “a” or “an” may mean one or more. As used herein in the claim(s), when used in conjunction with the word “comprising”, the words “a” or “an” may mean one or more than one. As used herein “another” or “other” may mean at least a second or more of the same or different claim element or components thereof. The following abbreviations have been used herein: 5-LO: 5-Lipoxygenase, ATV: Atorvastatin, and PIO: Pioglitazone.

The drugs described herein may be administered independently one or more times to achieve, maintain or improve upon a therapeutic effect. It is well within the skill of an artisan to determine dosage or whether a suitable dosage of the composition comprises a single administered dose or multiple administered doses. An appropriate dosage depends on the subject's health, attainment of the required effect of the drug (for instance, prevention of formation of blood clots, prevention of inflammation, etc) and the formulation used.

Additionally, although the present invention has demonstrated the effects of statins such as atorvastatin and thiazolidinedione such as pioglitazine, the present invention contemplates that other compound belonging to this group will also exhibit similar effects. Hence, examples of statins may include but is not limited to atorvastatin, and the examples of thiazolidinediones may include but is not limited to pioglitazone. Similarly, although the present invention has demonstrated the effect of cilostazol in activating protein kinase A by increasing cAMP levels, the present invention contemplates other compounds which increase cAMP levels may also activate protein kinase A. Hence, such compounds may include but is not limited to cilostazol, sitagliptin. Accordingly, the these compounds will also exhibit similar synergistic effects.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLE 1

### Materials

H-89, monoclonal anti-b Actin antibodies and monoclonal anti-myosin antibodies were purchased from Sigma (St. Louis, Mo.), anti-5-lipoxygenase and anti-Ser⁵²³ phosphorylated 5-lipoxygenase antibodies, polyclonal anti-COX2 antibodies and LTB4 EIA kit from Cayman Chemicals (Ann Arbor, Mich.), and anti-cPLA2 antibodies from Cell Signaling Technology (Danvers, Mass.). DAPI was purchased from Vector Laboratories (Burlingame, Calif.), goat anti-mouse Alexa 488 antibodies from Molecular Probes (Eugene, Oreg.), and Universal Negative Controls for Mouse and Rabbit IgG from DAKO Corporation (Carinteria, Calif.). ELISA kit for 15ELXA was purchased from Oxford Biomedical Research (Oxford, Mich.). PIO was provided by Takeda Pharmaceuticals North America, Inc. (Lincolnshire, Ill.) and ATV by Pfizer Pharmaceuticals (New York, N.Y.).

### EXAMPLE 2

### Animals

Male Sprague-Dawley rats received humane care in compliance with ‘The Guide for the Care and Use of Laboratory Animals’ published by the US National Institutes of Health [NIH Publication No. 85-23, revised 1996].

### EXAMPLE 3

### In-vivo Experiment

Rats received: 1) PIO (10 mg/kg/d); 2) ATV (10 mg/kg/d); 3) PIO (10 mg/kg/d)+H89 (20 mg/kg); 4) ATV (10 mg/kg/d)+H89 (20 mg/kg); 5) H89 (20 mg/kg) or 6) water alone (control). PIO and ATV were suspended in water and administered by gastric gavage once daily for 3 days; H-89 was dissolved in DMSO (final concentration 5% v/v) and injected intraperitoneally on the third day. Rats in groups 5 and 6 received water by gastric gavage once daily for 3 days. Rats in groups 1, 2 and 6 received intraperitoneal inkection of DMSO 5%. Sixteen hours after the injection, rats were euthanized and the hearts explanted for further analyses. In another experiment, rats received 3-day pretreatment with: 1) water (sham); 2) PIO (10 mg/kg/d); 3) ATV (10 mg/kg/d); 4) PIO (10 mg/kg/d)+ATV (10 mg/kg/d); or 5) LPS (10 mg/kg).

PIO and ATV were administered by oral gavage once daily as above; LPS was administered intravenously. In addition, rats in groups 1-4 received intravenous saline on the fourth day, whereas rats in group 1 and 5 received water by gastric gavage once daily for 3 days and the LPS injection on the third day. Sixteen hours after the injection, rats were euthanized and the hearts explanted for further analyses.

### EXAMPLE 4

### In-vitro Experiment

Cardiac myocytes were isolated from adult Sprague-Dawley rats (250-300 g, male). Animals were heparinized (1,000-2,000 units i.p.) 5 min before being anesthetized with ketamine (100 mg/kg) and xylazine (10 mg/kg), and the hearts were removed and placed in ice-cold heart media solution (in mmol/l: 112 NaCl, 5.4 KCl, 1 MgCl2, 9 NaH2PO4, and 11.1 D-glucose; supplemented with 10 HEPES, 30 taurine, 2 DL-carnitine, and 2 creatine, pH. 7.4). The hearts were perfused retrogradely in a Langendorff apparatus with Ca²⁺-free heart media for 5 min at 5 ml/min at 37° C., followed by perfusion with Ca²⁺-free heart media containing collagenase II, 210 U/mg (Worthington, Lakewood, N.J.) for 20 min. After perfusion, both ventricles were removed from the heart and minced in collagenase II-containing heart media for 10-15 min. The cell solution was then washed several times to remove collagenase II and reexposed to 1.2 mM Ca2⁺ over 25 min to produce Ca2⁺-tolerant cardiac myocytes. Myocytes were then plated in 4% FBS on laminin (2 μg/cm²)-coated plates for 1 h and incubated at 37° C. in 5% CO2 for 12-24 h before experiments (21). Cells were incubated with 1) vehicle (0.07% ethanol); 2) PIO (10 μM)+ATV (10 μM); 3) H-89 (0.1 μM); or 4) PIO+ATV+H-89 for 12 hours. The supernatants were collected directly for LTB4 analyses by ELISA and the cells were harvested for immunoblotting. In addition, cells were plated in 8-chamber slides, received the same treatment as above and were used for immunohistochemical staining.

### EXAMPLE 5

### ELISA

The hearts were rapidly explanted, rinsed in cold PBS (pH 7.4), containing 0.16 mg/ml heparin to remove red blood cells and clots, frozen in liquid nitrogen and stored at −70° C. Myocardial samples from the anterior left ventricular wall were homogenized in ethanol (5 ml/g) and centrifuged at 10,000 g×15 min at 4° C. The supernatant was diluted with water and acidified to pH 3.5 with 1M HCl. The sample was loaded into C-18 Sep-Pak light column (Waters Corporation, Milford, Mass.) and washed with 1 ml of water followed by 1 ml of petroleum ether. The sample was eluted with 2 ml of methyl formate. The methyl formate was evaporated with N₂ and the residue was dissolved in extraction buffer. the manufacturer instruction for the 15ELXA and LTB4 immunoassay kits were followed.

### EXAMPLE 6

### Separation of the Cytosolic, Membranous and Nuclear Fraction

Myocardial samples (0.25 g) were homogenized, mixed with Buffer A Mix [Hepes (pH 7.9) 10 mM, KCl 10 mM, EDTA 10 mM, DTT 100 mM, protease inhibitor cocktail, and IGEPAL 10%, (Sigma, St Lois, Mo.)], homogenized again and incubated for 15 min on ice, and centrifuged at 850×g for 10 min at 4° C. The supernatant was discharged, Buffer A Mix was added again and the samples incubated for an additional 15 min on ice, and centrifuged at 15,000×g for 3 min at 40 C. The supernatant contains the cytosolic fraction was collected. The pellet was resuspended in 150 μl of Buffer B Mix [Hepes (pH 7.9) 20 mM, NaCl 0.4M, EDTA 1 mM, glycerol 10%, protease inhibitor cocktail, and IGEPAL 10%], the tubes were shaked on ice at 200 rpm for 2 h, centrifuged at 15,000×g for 5 min at 4° C., and supernatants were collected as the nuclear fraction. The cytosolic and nuclear fractions were used for immunoblotting for 5LO.

### EXAMPLE 7

### Immunoblotting

The hearts were rapidly explanted, rinsed in cold PBS (pH 7.4), containing 0.16 mg/ml heparin to remove red blood cells and clots, frozen in liquid nitrogen and stored at −70° C. Myocardial samples from the anterior left ventricular wall were homogenized in RIPA lysis buffer (Santa Cruz Biotechnology, Santa Cruz, Calif.) and centrifuged at 14,000 rpm for 15 min at 4° C. The supernatant was collected and the total protein concentration was determined using the Lowry protein assay. For immunoblotting of total 5LO in the in-vivo experiment the cytosolic and nuclear fraction, separately was used. The protein samples with loading buffer were run in 4-20% Tri-HCl Ready Gel at a 100V for 2 h until the desired molecular weight bands were separated. After electrophoresis, the gel was equilibrated in transfer buffer (25 mM Tris, 193 mM glycine, 0.1% SDS and 10% methanol) and the proteins were transferred to nitrocellulose membrane. The protein signals were quantified by an image-scanning-densitometer and the strength of each protein signal was normalized to the corresponding b-actin stain signal. Data are expressed as a ratio between the protein and the corresponding b-actin signal density.

### EXAMPLE 8

### Immunohistochemical Study

Immunofluorescent labeling was performed on paraffin sections (5-μm) of 4% formaldehyde-fixed rat cardiac tissue, as described previously (22). The primary antibodies were mouse anti-myosin IgG, rabbit anti-COX2 IgG and rabbit anti-5-lipoxygenase IGG, and diluted in 1:2000, 1:1000, and 1:2000, respectively. The secondary antibodies were goat anti-mouse Alexa 488 (diluted in 1:500) for mouse primary antibody and goat anti-rabbit Alexa 594 (diluted in 1:500) for rabbit primary antibodies. Slides were counterstained with DAPI and mounted with Cytoseal XYL mounting medium. The specificity of mouse and rabbit primary antibodies was tested by substituting them with Universal Negative Controls for Mouse and Rabbit IgG. All the slides were viewed under an Olympus BX51 microscope [images recorded by a DP70 Digital camera (Olympus Optical Co., Ltd., Tokyo, Japan)] or confocal microscope (Bio-Rad 2100 (Hercules, Calif.).

### EXAMPLE 9

### Co-immunoprecipitation

For co-immunoprecipitation, myocardial cytosolic, membranous and nuclear fractions (500 μg) were incubated with anti-5LO antibodies for 4 h followed by overnight incubation at 4° C. with Protein-A-Agarose. The agarose beads were collected by centrifugation and SDS/PAGE Western immunoblotting was performed with the supernatant fraction. The anti-5LO precipitates were subjected to immunoblotting with anti-COX2 or anti-cPLA₂ antibodies.

### EXAMPLE 10

### Real-time PCR

Equal amounts of total cellular RNA were reverse-transcribed with oligo(dT) primer by use of AMV Reverse Transcriptase (Applied Biosystems). Transcribed cDNAs (40 ng) were used for Real Time PCR with specific primers: rat ALOX5. (ALOX5F: AGCCAACAAGATTGTTCCCATCGC (SEQ ID NO: 1) AlOX5R: TGGCAATACCGAACACCTCAGACA (SEQ ID NO: 2)), and rat glyceraldehyde-3-phosphate dehydrogenase (GAPDH)(5′-ACCCCCAATGTATCCGTTGT-3′ (SEQ ID NO: 3), 5′-TACTCCTTGGAGGCCATGTA-3′ (SEQ ID NO: 4)). The Ct (threshold cycle) is defined as the number of cycles required for the fluorescence signal to exceed the detection threshold. Expression of the ALOX5 relative to the GAPDH was calculated as the different between the threshold values of these two genes. Melting curve analysis was performed during real-time PCR to analyze and verify the specificity of the reaction. The amount of target (2^{-ΔΔCT}) was obtained by normalized to endogenous reference (GAPDH) and relative to a calibrator (average of the control samples). The values was given as the means±S.E. of four independent experiments. As positive controls we used rat White Blood cells from a rat at baseline and 16 h after intravenous injection of LPS (10 mg/kg).

### EXAMPLE 11

### N Vivo Studies:

At first, immunoblotting was used to assess the effect of PIO and ATV, alone or with H-89 in myocardial levels of total 5LO and P-5LO in the whole cell homogenate (FIG. 1). PIO and ATV did not affect total 5LO concentration, but they increased myocardial levels of Ser⁵²³ phosphorylated 5LO. H-89 alone did not affect total 5LO or P-5LO levels, however, it completely blocked the increase in P-5LO by both PIO and ATV. The effects of PIO and ATV were compared to that of LPS on 5LO phosphorylation (FIG. 2). PIO and ATV alone or in combination, caused a significantly greater increase in 5LO phospholylation than LPS.

For further characterization of the effects of PIO and ATV on 5LO expression and translocation, the immunoblotting was performed separately in the cytosolic, membranous and nuclear fractions of the myocardial cells (FIG. 3). PIO and ATV caused a small, yet significant increase in 5LO levels in the cytosolic fraction. In contrast, they had no detectable effect on the 5LO levels in the membranous fraction. H-89 alone had no significant effect on 5LO levels in both the cytosolic and membranous fractions. On the other hand, when H-89 was combined with either PIO or ATV, there were significant decreases in 5LO levels in the cytosolic fraction and significant increases in the membranous fraction, suggesting translocation of 5LO from the cytosolic fraction to the membranous fraction. There was no expression of 5LO in the nuclear fraction in all groups studied.

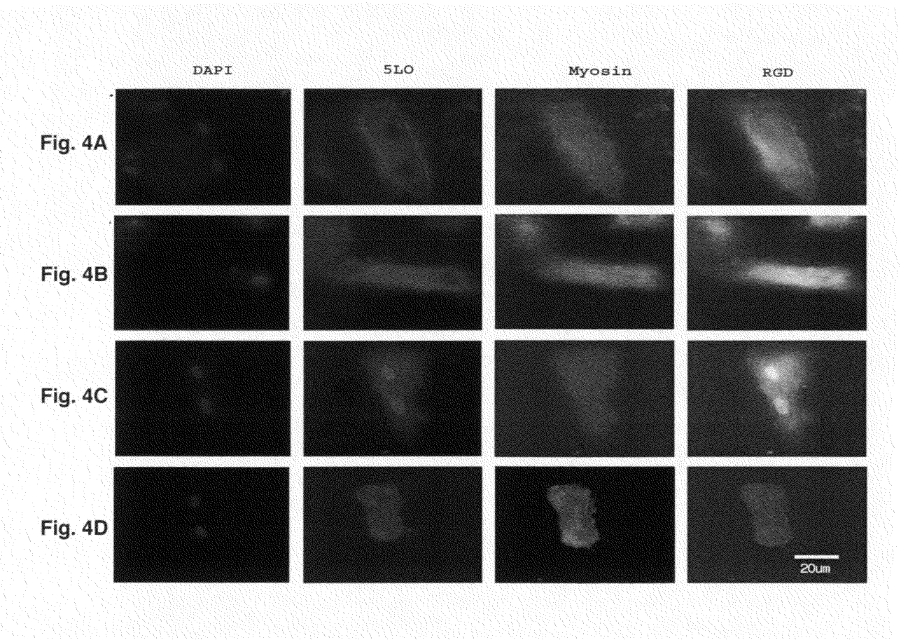

Subsequently, immunofluorescence was used to determine localization of 5LO in-vivo in myocardial tissue of rats pretreated with LPS, PIO+ATV or vehicle alone. PIO+ATV caused enhanced staining of 5LO in the cytoplasm of myocytes, whereas LPS caused migration of 5LO to the perinuclear membrane without an apparent increase in overall intensity (FIG. 4).

### In Vitro Studies:

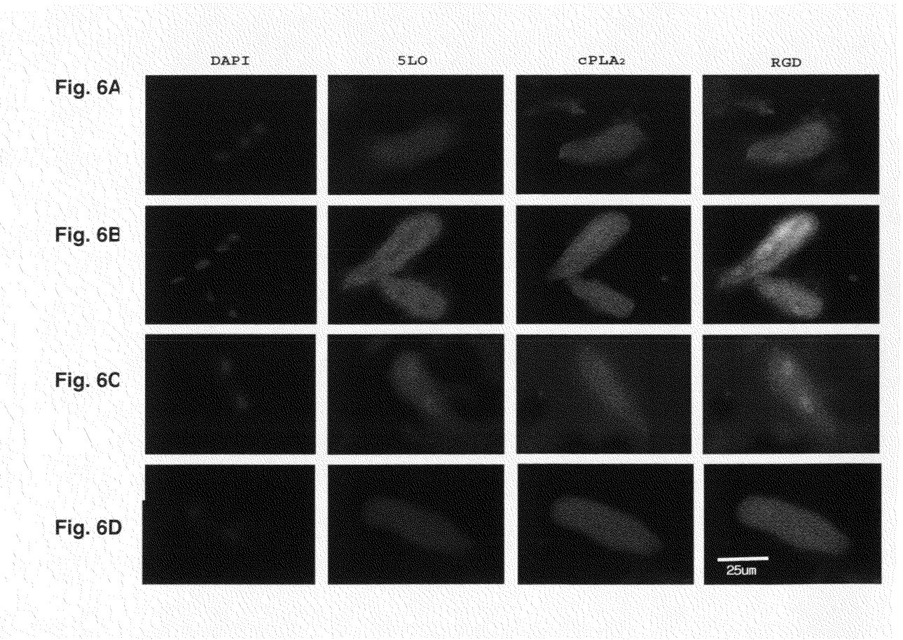

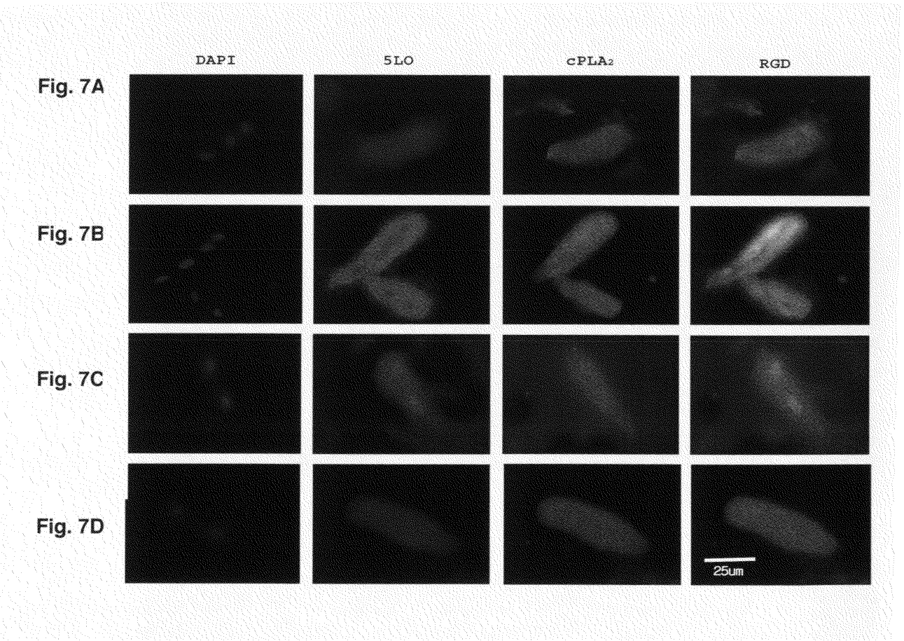

The 5LO localization in rat cardiomyocyte cultures was further characterized after incubation with PIO+ATV in the presence and absence of H-89, a specific PKA inhibitor. 5LO was expressed in the cytoplasm of myosin-positive cells (FIG. 5). PIO+ATV also enhanced 5LO staining in cytoplasm. H-89 alone had no effect on 5LO expression or distribution; however, when combined with PIO+ATV there was a shift of 5LO to the nucleus. Confocal microscopy (×120 magnification) shows that in the PIO+ATV+H-89 treated cells, 5LO is localized around, but not inside the nucleus (FIG. 6). PIO+ATV increased the expression of COX2 in the cytoplasm (FIG. 7), and H-89 had no effect on this enhancement, but did cause a shift of 5LO to the nuclear membrane.

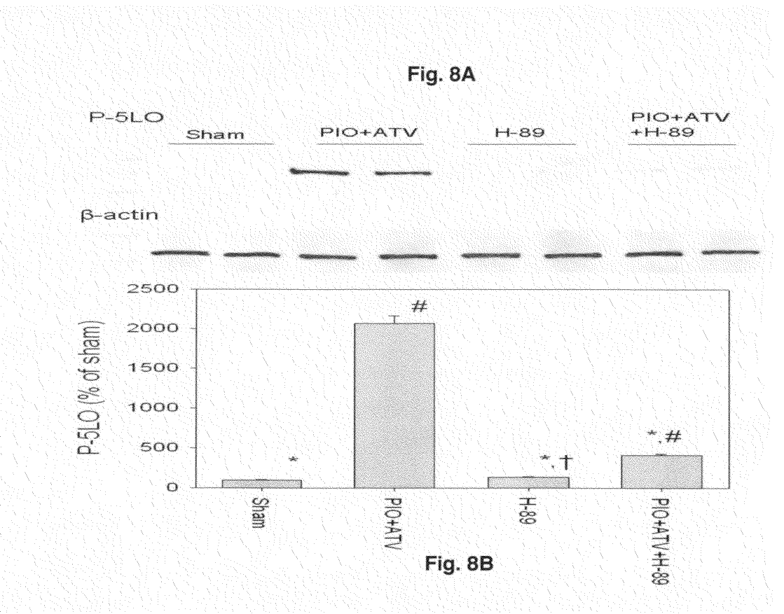

PIO+ATV had no affect on total 5LO levels (data not shown), but did increase P-5LO levels in the cell cultures (FIG. 8). The 5LO phosphorylation was almost completely blocked by H-89. PIO+ATV increased the levels of 15ELXA. H-89 alone had no effect; however, it attenuated the PIO+ATV augmentation of 15ELXA levels (FIG. 9A). Finally, whereas PIO+ATV alone and H-89 alone had no significant effect on LTB4, H-89 given together with PIO+ATV, significantly increased myocardial LTB4 levels (FIG. 9B).

### Immuno-precipitation:

Using whole cell lysate, it is demonstrated herein that in the control animals there was no co-immunoprecipitation of either COX2 or cPLA₂. However, in rats treated with either PIO or ATV, COX2, but not cPLA₂, precipitated with 5LO. In contrast, in rats treated with PIO or ATV in combination with H-89, 5LO precipitated with cPLA2, but not with COX2 (FIG. 10).

To further characterize the location of these interactions, the co-immunoprecipitation were performed in the cytosolic and membranous fractions of the same hearts (FIG. 11). Co-immunoprecipitation of 5LO with COX2 in the ATV or PIO treated rats occurred only in the cytosolic fraction. In contrast, the interaction between cPLA₂ and 5LO in the rats treated with H-89 combined with PIO or ATV occurred in the membranous fraction.

### Rt-PCR:

To confirm that adult rat myocardial cells expressed 5LO, rtPCR was used herein. White blood cells isolated from rat blood at basal condition (control) or 16 h after stimulation with 5LO served as positive controls [24]. Both adult rat cardiomyocytes and white blood cells express mRNA for 5LO (FIG. 12). PIO, ATV and H-89 alone or in combination did not affect 5LO mRNA levels in the cardiomyocytes. LPS increased 5LO expression in the white blood cells.

### EXAMPLE 12

### Synergistic Effects of a Protein Kinase A Activator and Atrovastatin on Myocardial Protection

The effects of cilostazol (Pletal) and atorvastatin on myocardial protection was measured in a rat infarct model. Pletal is a phosphodiesterase III inhibitor, thus increasing intracellular cAMP levels. cAMP activates PKA. ATV at 2 mg/kg/d had no effect. Cilostazol 20 mg/kg/d reduced infarct size. However, when combined with ATV 2 mg/kg/d the effect was much greater. There are 8 rats in the control and ATV 2 mg/kg/d groups and 6 rats in the cilostazol and the combination groups. Infarct size in the ATV 2 mg/kg/d (30.48±1.46%) is not different from the controls (33.97±2.76%). The cilostazol group(15.47±1.61%) is significantly smaller than the controls (p<0.001). The combination (4.31±0.48%) is significantly different (<0.001) from the controls and ATV 2 mg/kg and (=0.006) versus the cilostazol alone group.

The following references are cited herein:
1. Agarwal, R. (2006) Am J Physiol Renal Physiol 290(3), F600-605
2. Shimizu et al. (2006) Diabet Med 23(3), 253-257
3. Marketou, et al. (2006) Angiology 57(2), 211-218
4. Sola et al (2006) J Am Coll Cardiol 47(2), 332-337
5. Birnbaum, et al. (2006) Circulation 114(9), 929-935
6. Ariel et al (2003) J Immunol 170(12), 6266-6272
7. Hachicha et al (1999) J Exp Med 189(12), 1923-1930
8. Leonard et al. (2002) J Am Soc Nephrol 13(6), 1657-1662
9. Pouliot, M., and Serhan, C. N. (1999) J Periodontal Res 34(7), 370-373
10. Radmark, O., and Samuelsson, B. (2005) Biochem Biophys Res Commun 338(1), 102-110
11. Peters-Golden, and McNish, (1993) Biochem Biophys Res Commun 196(1), 147-153
12. Pouliot, et al. (1996) Eur J Biochem 238(1), 250-258
13. Luo, et al. (2005) J Biol Chem 280(49), 40609-40616
14. Harris, et al (2004) Am J Physiol Heart Circ Physiol 287(2), H560-566
15. Shah, D. I., and Singh, M. (2006) Endothelium 13(4), 267-277
16. Manickavasagam, et al (2007) Cardiovascular Drugs and Therapy 21(5), 321-330
17. Birnbaum, et al. (2005) Cardiovasc Res 65(2), 345-355
18. Atar, et al (2006) Am J Physiol Heart Circ Physiol 290(5), H1960-1968
19. Ye, et al (2006) Am J Physiol Heart Circ Physiol 291(3), H1158-1169
20. Kim, et al (2005) Science 310(5756), 1966-1970
21. Birnbaum, et al. (In press) Prostaglandins Other Lipid Mediat
22. Patel et al (2006) Am J Physiol Heart Circ Physiol 291(1), H344-350
23. Huang, et al. (2005) Am J Physiol Heart Circ Physiol 288(2), H497-503
24. Surette, et al. (1998) Faseb J 12(14), 1521-1531
25. Peters-Golden and Henderson, (2007) New England jour of Medicine 357(18), 1841-1854
26. Linde, et al (2007) Cardiovascular research 73(1), 26-36
27. Wilson et al. (2004) Journal of molecular and cellular cardiology 37(4), 801-811
28. Fredj, et al (2005) Journal of cellular physiology 202(3), 891-899
29. Massey, et al (1992) The American journal of physiology 263(2 Pt 1), C494-501
30. Przygodzki, et al (2005) Biochimica et biophysica acta 1740(3), 481-488
31. Liu, P et al. (2003) American journal of physiology 284(4), H1269-1276
32. Kuzuya, et al (1993) Cardiovascular research 27(6), 1056-1060
33. Birnbaum, et al (2007) Prostaglandins & Other Lipid Mediators 83(1-2), 89-98
34. Parfenova, et al (2001) Am J Physiol Cell Physiol 281(1), C166-178
35. Parfenova, et al (1997) The American journal of physiology 273(1 Pt 1), C277-288
36. Steinhilber, D. (1994) Pharm Acta Helv 69(1), 3-14
37. Rouzer, et al (1990) J Biol Chem 265(3), 1436-1442
38. Dwyer, et al (2004) The New England journal of medicine 350(1), 29-37
39. Hakonarson, et al (2005) Jama 293(18), 2245-2256
40. Helgadottir, et al (2004) Nat Genet 36(3), 233-239
41. Lohmussaar, et al (2005) Stroke 36(4), 731-736
42. Helgadottir, et al. (2005) Am J Hum Genet 76(3), 505-509
43. Topol, et al. (2006) Hum Mol Genet 15 Spec No 2, R117-123
44. Kajimoto, et al. (2005) Circ J 69(9), 1029-1034
45. Helgadottir, et al (2006) Nat Genet 38(1), 68-74
46. Flamand, N., Lefebvre, J., et al, (2006) J Biol Chem 281(1), 129-136
47. Goodman, et al (2004) Carcinogenesis 25(12), 2467-2472
48. Manev, H., and Manev, R. (2006) Med Hypotheses 66(3), 501-503
49. Qu, et al. (2001) J Neuropsychiatry Clin Neurosci 13(2), 304-305
50. Manev, H. (2000) Med Hypotheses 54(1), 75-76
51. Drazen, et al (1999) Nat Genet 22(2), 168-170
52. Poynter, et al (2005) N Engl J Med 352(21), 2184-2192
53. Sparks, et al (2006) Acta Neurol Scand Suppl 185, 78-86
54. Cittelly, et al (2005) J. Neurotrauma 22(10), 1164
55. Simizu, et al (2004) Cancer Sci 95(3), 266-270

What is claimed is:

1. A method of attenuating a pro-inflammatory state specific for a disease in an individual comprising:
administering a pharmacologically effective dose of a compound(s) that phosphorylates 5-lipoxygenase, prevents translocation of the 5-lipoxygenase from cytosol to the peri-nuclear membrane or both, thereby attenuating the pro-inflammatory state specific for the disease in the individual.

2. The method of claim 1, wherein said 5-lipoxygenase is phosphorylated at serine-523 residue of 5-lipoxygenase.

3. The method of claim 1, wherein said cytosolic phosphorylated 5-lipoxygenase interacts with Cox-2 to produce 15-epilipoxin-A4.

4. The method of claim 1, wherein said compound directly or indirectly activates protein kinase A such that said activated protein kinase A phosphorylates 5-lipoxygenase.

5. The method of claim 4, wherein said compound is a HMGCoA Reductase inhibitor, Atorvastatin or PPAR-g agonist, pioglitazone, sitagliptin, or a combination of thereof.

6. The method of claim 1, wherein the pro-inflammatory effect is due to absence of phosphorylation on serine-523, translocation of 5-lipoxygenase to the nuclear membrane, metabolism of arachidonic acid into leukotriene B4 or a combination thereof.

7. The method of claim 1, wherein said disease state is artherosclerosis, arthiritis, asthma, cancer, stroke, myocardial infarction or Alzheimers.

8. A method of decreasing the risk or progression of a disease in an individual comprising:
administering a pharmacologically effective dose of a compound that inhibits the production of Leukotriene-B₄ to said individual, thereby decreasing the risk or progression of a disease in the individual.

9. The method of claim 8, wherein said administration of the compound results in direct or indirect activation of protein kinase A.

10. The method of claim 9, wherein said activation of protein kinase A results in phosphorylation of serine-523 residue of 5-Lipoxygenase.

11. The method of claim 10, wherein said phosphorylation prevents the localization of 5-Lipoxygenase from cytosol to the perinuclear membrane.

12. The method of claim 11, wherein said prevention of perinuclear localization results in decreased leukotriene-B₄ production and an increased 15-epilipoxin-A4 production.

13. The method of claim 8, wherein said compound being administered is a HMGCoA Reductase inhibitor, Atorvastatin or the PPAR-gagonist, Pioglitazone, sitagliptin, or a combination thereof.

14. The method of claim 8, wherein said disease state is artherosclerosis, arthritis, asthma, cancer, stroke, myocardial infarction or Alzheimer's.

15. A method of augmenting anti-inflammatory effects in an individual in need of such augmentation, comprising:
administering a pharmacologically effective dose of a compound that that phosphorylates serine-523 residue of 5-Lipoxygenase such said phosphorylation of 5-Lipoxygenase regulates the production of anti-inflammatory and pro-inflammatory metabolites of arachidonic acid, thereby augmenting the anti-inflammatory effects in said individual.

16. The method of claim 15, wherein said compound phosphorylates serine-523 residue of 5-Lipoxygenase by directly or indirectly activating protein kinase A.

17. The method of claim 16, wherein said phosphorylation of 5-Lipoxygenase prevents the localization of 5-Lipoxygenase to the perinuclear membrane resulting in interaction of phosphorylated 5-lipoxygenase with COX2 and production of anti-inflammatory metabolite of arachidonic acid and inhibition of pro-inflammatory metabolite of arachidonic acid.

18. The method of claim 17, wherein said pro-inflammatory metabolite of arachidonic acid is Leukotriene B₄ and wherein said anti-inflammatory metabolite of arachidonic acid is 15-epilipoxin-A₄.

19. The method of claim 18, wherein said 15-epilipoxin-A₄ produced mediates anti-inflammatory effects by inhibiting the production of IL-6 and TNF-a.

20. The method of claim 15, wherein said compound being administered is a HMGCoA Reductase inhibitor, Atorvastatin or PPAR-g agonist, Pioglitazone, sitagliptin, or a combination thereof.

21. The method of claim 15, wherein said individual in need of augmentation of anti-inflammatory effect is suffering from artherosclerosis, arthritis, asthma, cancer, stroke, myocardial infarction or Alzheimers.

22. A method for screening for a drug useful for augmenting anti-inflammatory effects in a disease state comprising:
contacting a sample peptide comprising the serine-523 residue of 5-Lipoxygenase with a test compound;
providing the necessary enzymes and ATP, and
determining the effect of the compound on the phosphorylation of the serine-523 residue, wherein phosphorylation of the serine-523 residue of the peptide in the presence of the test compound indicates that the test compound is the drug useful for augmenting anti-inflammatory effects in said disease state.

23. The method of claim 22, wherein said drug is an activator of protein kinase A, prevents localization of 5-lipoxygenase to the peri-nuclear membrane or a combination thereof.

24. The method of claim 23, wherein said disease state is artherosclerosis, arthiritis, asthma, cancer, stroke, myocardial infarction or Alzheimers.

25. A method of ameliorating the side effects of statin therapy in an individual comprising:
administering pharmacologically effective amounts of of a protein kinase A activator in combination with statins and/or thiazolidinediones, wherein said administration ameliorates the side-effects of statin therapy in said individual.

26. The method of claim 25, wherein said side effects comprise muscle aches and/or elevation of muscle and/or liver enzymes.

27. The method of claim 33, wherein said administration of protein kinase A activator leads to phosphorylation of serine-523 residue on 5-lipoxygenase and prevents translocation of said phosphorylated 5-Lipoxygenase from the cytosol to perinuclear membrane such that said phosphorylated cytosolic 5-Lipoxygenase interacts with COX-2, producing 15-epilipoxin A4 and inhibiting the production of Leukotriene-B4.

28. The method of claim 27, wherein said production of the 15-epilipoxin-A₄ inhibits the production of IL-6 and TNF-a.

29. The method of claim 25, wherein said individual on statin therapy is suffering from artherosclerosis, arthritis, asthma, cancer, stroke, myocardial infarction or Alzheimer's.

30. A method of inducing myocardial protection in an individual comprising:
administrating pharmacologically effective amounts of a protein kinase A activator in combination with statins and/or thiazolidinediones, wherein said administration synergistically reduces the infarct size, thereby inducing myocardial protection in the individual.

31. The method of claim 30, wherein said protein kinase A activator increases the intracellular levels of cyclic adenosine monophosphate (cAMP) such that said increased cAMP levels activate protein kinase A.

32. The method of claim 31, wherein the protein kinase A activator is cilostazol or sitagliptin.

33. The method of claim 30, wherein said statin is Atrovastatin.

34. The method of claim 30, wherein said thiazolidinedione is pioglitazone.

35. The method of claim 30, wherein said individual is suffering from a myocardial infarction.
